(19) United States
(12) Patent Application Publication
(10) Pub. No.: US 2004/0214285 A1
(43) Pub. Date: Oct. 28, 2004

(54) HIV-specific fusion proteins and therapeutic and diagnostic methods for use

(76) Inventors: David J. Glass, Cortlandt Manor, NY (US); Margaret Karow, Putnam Valley, NY (US); Eric Smith, New York, NY (US)

Correspondence Address: REGENERON PHARMACEUTICALS, INC, 777 OLD SAW MILL RIVER ROAD, TARRYTOWN, NY, 10591, US

(21) Appl. No.: 10/768,932

(22) Filed: Jan. 30, 2004

Related U.S. Application Data
(60) Provisional application No. 60/446,347, filed on Feb. 10, 2003.

(51) Int. Cl. C07K 14/705; C07H 21/04; A61K 39/12

(52) U.S. Cl. 435/69.3; 435/320.1; 435/325; 530/350; 536/23.5; 424/186.1

(57) ABSTRACT

A HIV-specific fusion polypeptide, comprising (a) one or more domains which comprise a cellular co-receptor protein, or a fragment, derivative, or functional equivalent thereof; (b) one or more domains which comprise a cellular receptor protein, or a fragment, derivative, or functional equivalent thereof; and optionally (c) a multimerizing component, and (d) one or more domains of a viral protein, or a fragment or derivative thereof. In specific embodiments, the HIV-specific fusion protein is a multimer capable of binding an HIV particle, and is useful for the treatment of HIV infections.

### CROSS-REFERENCE TO RELATED APPLICATIONS

This application claims the benefit under 35 USC § 119(e) of U.S. Provisional 60/446,347 filed 10 Feb. 2003, which application is herein specifically incorporated by reference in its entirety.

### REFERENCE TO SEQUENCE LISTING

This application refers to sequences listed in a Sequence Listing hereinto attached, which is considered to be part of the disclosure of the invention.

### BACKGROUND OF THE INVENTION

1. Field of the Invention

This invention relates to HIV-specific fusion proteins with increased affinity for a viral target molecule, methods of producing such HIV-specific fusion proteins, and methods for inactivating viruses. More specifically, the invention provides HIV-specific fusion proteins useful for inactivating the Human Immunodeficiency Virus (HIV) and for treating or preventing Acquired Immune Deficiency Syndrome (AIDS).

2. Description of Related Art

The incidence of Acquired Immune Deficiency Syndrome (AIDS), resulting from infection with the Human Immunodeficiency Virus (HIV) continues to increase. The initial events in infection of human T lymphocytes, macrophages, and other cells by HIV have been elucidated. These events involve the attachment of the HIV envelope glycoprotein gp120 to a cell by binding to the cellular receptor, CD4, and a co-receptor (usually CCR5, but also CXCR4, and perhaps others). Lectin binding receptors, such as DC-SIGN, also mediate the presentation and transmission of the viruses. Approaches in developing a therapeutic for AIDS include development of bispecific molecules which can bind a pathogen and/or target the pathogen for destruction by effector cells (U.S. Pat. No. 5,897,861), and immunoadhesin molecules containing portions of CD4 fused to the constant region of antibody light and heavy chains (Capon et al. (1989) Nature 337:525-531).

### BRIEF SUMMARY OF THE INVENTION

The present invention provides a HIV-specific fusion protein capable of binding the Human Immunodeficiency Virus (HIV). Binding of a multi specific protein capable of binding an HIV particle (also termed an “HIV trap” or an “HIV-specific fusion protein”) prevents or inhibits the virus from cell entry. Accordingly, the HIV-specific fusion proteins of the invention are useful for reducing, preventing, or inhibiting HIV infection and/or the progression of HIV infection to AIDS. The HIV-specific fusion proteins of the invention are further useful for detecting HIV in a variety of in vitro and in vivo diagnostic and prognostic assays.

Accordingly, in a first aspect the invention provides a HIV-specific fusion polypeptide comprising (i) one or more domains which comprise a cellular co-receptor protein, or a fragment or derivative capable of binding gp120, or functional equivalent thereof (“CCR”); (ii) one or more domains which comprise a cellular receptor protein, or a fragment or derivative capable of binding gp120, or functional equivalent thereof (“CR”); and optionally (iii) a fusion component (“FC”), and (iv) one or more domains of a viral protein, or a fragment, derivative, or functional equivalent thereof (“VP”).

In one embodiment, the HIV-specific fusion polypeptide comprises one or more CCR domains. In a specific embodiment, the co-receptor protein is human CCR5 or a fragment or derivative thereof. In a more specific embodiments, the domain of a CCR protein is the amino-terminal portion of the CCR5 protein. In another embodiment, CCR is human CXCR4, or a fragment or derivative thereof. In yet another embodiment, CCR is DC-SIGN, or a fragment thereof capable of binding gp120 of an HIV virus particle. When the HIV-specific fusion polypeptide of the invention comprises more than one CRR component, the CRR components may be the same or different.

In one embodiment, the HIV-specific fusion polypeptide of the invention comprises one or more CR domains. In a more specific embodiment, CR is human CD4, or a fragment or derivative thereof capable of binding gp120. CD4 has four immunoglobulin-like (Ig-like) domains numbered 1-4. Accordingly, in a more specific embodiment, the HIV-specific fusion protein comprises one or more CD4 Ig-like domains. In specific embodiments, the HIV-specific fusion polypeptide comprises Ig-like domain 1, 2, 3, and/or 4; in a preferred embodiment, the HIV-specific fusion polypeptide comprises Ig-like domain 1 of CD4 or Ig-like domains 1 and 2 of CD4. The one or more Ig-like CD4 domains may be modified, e.g., by mutation or deletion. See, for example, constructs described in Example 1 in which fusion polypeptides comprise domains 1 and 2 of CD4 in which 10 amino acids of the N-terminus of Ig 1 are deleted. In another embodiment, the receptor protein is DC-SIGN, or a fragment thereof capable of binding gp120. When the HIV-specific fusion polypeptide of the invention comprises more than one CR component, the CR components may be the same or different.

The HIV-specific fusion polypeptide of the invention optionally includes a fusion component which is a component that enhances the functionality of the fusion polypeptide. Thus, for example, a fusion component may enhance the biological activity of the fusion polypeptide, aid in its production and/or recovery, or enhance a pharmacological property or the pharmacokinetic profile of the fusion polypeptide by, for example, enhancing its serum half-life, tissue penetrability, lack of immunogenicity, or stability. In preferred embodiments, the fusion component is one or more components selected from the group consisting of a multimerizing component, fusion partner, a targeting protein, a serum protein, or a molecule capable of binding a serum protein.

When the fusion component is a multimerizing component, it includes any natural or synthetic sequence capable of interacting with another multimerizing component to form a higher order structure, e.g., a dimer, a trimer, etc. The term “HIV-specific fusion protein” includes higher order complexes composed of more than one fusion polypeptide and capable of binding an HIV viral particle. In specific embodiments a multimerizing component, may be selected from the group consisting of (i) an immunoglobulin-derived domain, (ii) a cleavable region (C-region), (ii) an amino acid sequence between 1 to about 500 amino acids in length, optionally comprising at least one cysteine residue, (iii) a leucine zipper, (iv) a helix loop motif, and (v) a coil-coil motif. In some embodiments, the multimerizing component comprises an immunoglobulin-derived domain from, for example, human IgG, IgM or IgA. In specific embodiments, the immunoglobulin-derived domain may be selected from the group consisting of the Fc domain of IgG, the heavy chain of IgG, and the light chain of IgG. The Fc domain of IgG may be selected from the isotypes IgG1, IgG2, IgG3, and IgG4, as well as any allotype within each isotype group. In one example of the HIV-specific fusion polypeptide of the invention, the fusion component is human FcΔ1(a).

The HIV-specific fusion polypeptide of the invention optionally includes a domain which is a viral protein or a fragment thereof. More specifically, the viral protein is a viral receptor. Even more specifically, the viral receptor is the HIV receptor gp41. Still more specifically, the viral protein is a fragment of the second helical region of gp41. Even more specifically, the fragment may be a peptide sequence comprising 15-15 amino acids of the C-terminal sequence of the gp41 protein. In one embodiment, the peptide is T20 or T-1249 (Trimeris Inc., Durham, N.C.).

In one embodiment, the component domains of the HIV-specific fusion polypeptide of the invention are connected directly to each other. In other embodiments, a spacer sequence may be included between one or more components, which may comprise one or more molecules, such as amino acids. For example, a spacer sequence may include one or more amino acids naturally connected to the domain component. A spacer sequence may also include a sequence used to enhance expression of the fusion polypeptide, provide restriction sites, allow component domains to form optimal tertiary structures and/or to enhance the interaction of a component with its target molecule. In one embodiment, the HIV-specific fusion polypeptide of the invention comprises one or more peptide sequences between one or more component domains which is(are) between 1-25 amino acids.

Further embodiments may include a signal sequence at the beginning or amino-terminus of a HIV-specific fusion polypeptide of the invention. Such a signal sequence may be native to the cell, recombinant, or synthetic.

The components of the HIV-specific fusion polypeptide of the invention may be arranged in a variety of configurations. For example, in certain embodiments, described from the beginning or amino-terminus of the fusion polypeptide, one or more cellular co-receptor domain(s) (CCR) may be followed by one or more cellular receptor domain(s) (CR), followed by a fusion component (M), optionally followed by one or more viral protein domain(s) (VP) at the carboxy-terminal end of the fusion polypeptide. Such a fusion polypeptide may also optionally include a signal sequence (SS) prior to the one or more cellular co-receptor domain(s).

Further configurations contemplated by the invention may be depicted as follows: (CCR)ₓ-(CR)_{y}-M; SS-(CCR)ₓ-(CR)_{y}-M; (CCR)ₓ-M-(CR)_{y}; SS-(CCR)ₓ-M-(CR)_{y}; (CCR)ₓ-M-(CR)_{y}-(VP)_{z}; (VP)_{z}-(CCR)ₓ-M-(CR)_{y}; SS-(CCR)ₓ-M-(CR)_{y}-(VP)_{z}; (CCR)ₓ-(CR)_{y}-M-(VP)_{z}; (VP)_{z}-(CCR)ₓ-(CR)_{y}-M SS-(CCR)ₓ-(CR)ₓ-M-(VP)_{z}; (CR)_{y}-(CCR)ₓ-M-(VP)_{z};(VP)_{z}-(CR)_{y}-(CCR)ₓ-M; (CCR)ₓ-(CR)_{y}-(CCR)ₓ-(CR)_{y}-M-VP)_{z}; SS-(CCR)ₓ-(CR)_{y}-(CCR)ₓ-(CR)_{y}-M-(VP)_{z}; (CR)_{y}-(CCR)ₓ-(CR)_{y}-(CCR)ₓ-M-(VP)_{z}; (VP)_{z}-(CR)_{y}-(CCR)ₓ-(CR)_{y}-(CCR)ₓ-M, (CR)_{y}-(CCR)ₓ-M-(CCR)ₓ, etc., wherein x≧1, y≧1, and z≧1. In a more specific embodiment, x=1-10, y=1-10, and z=1-10. In an even more specific embodiments, x=1, y 32 1, and z=1; or x=2, y=2, and z=1. Non-limiting exemplifications of the HIV-specific fusion polypeptides of the invention are provided in SEQ ID NOs: 1-9.

In a second aspect, the invention features a nucleic acid sequence encoding a HIV-specific fusion polypeptide, encoding (i) one or more domains which comprise a cellular co-receptor protein, or a fragment, derivative or functional equivalent thereof; (ii) one or more domains which comprise a cellular receptor protein, or a fragment, derivative or functional equivalent thereof; and optionally (iii) a fusion component, and (iv) one or more domains of a viral protein, or a fragment or derivative thereof.

In a related fourth aspect, the invention features a vector comprising the nucleic acid sequence of the invention. The invention further features an expression vector comprising a nucleic acid of the invention, wherein the nucleic acid molecule is operably linked to an expression control sequence. Also provided is a host-vector system for the production of a HIV-specific fusion polypeptide or protein of the invention which comprises the expression vector of the invention which has been introduced into a host cell suitable for expression of the HIV-specific fusion polypeptide or protein. Suitable host cells include, for example, bacterial cells, e.g., E. coli, yeast cells, e.g., Pichia pastoris, an insect cell, e.g., Spodoptera frugiperda, or a mammalian cell, such as CHO or COS.

In a related fifth aspect, the invention features a method of producing a HIV-specific fusion polypeptide of the invention, comprising culturing a host cell transfected with a vector comprising a nucleic acid sequence of the invention, under conditions suitable for expression of the protein from the host cell, and recovering the fusion polypeptide so produced. When the fusion polypeptide comprises a multimerizing component, the fusion polypeptides are generally recovered as dimeric or olimeric molecules, e.g., “HIV traps”formed via interaction of multimerizing components on separate fusion polypeptides.

In a sixth aspect, the invention features a multimeric HIV-specific protein comprised of two or more HIV-specific fusion polypeptides, wherein each HIV-specific fusion polypeptide comprises (i) one or more domains which comprise a cellular co-receptor protein, or a fragment, derivative, or functional equivalent thereof; (ii) one or more domains which comprise a cellular receptor protein, or a fragment, derivative , or functional equivalent thereof; and optionally (iii) a fusion component capable of acting as a multimerizing component, and (iv) one or more domains of a viral protein, or a fragment or derivative thereof. In one preferred embodiment, the multimeric protein of the invention is a dimer. In a specific embodiment, the multimeric protein is a dimer comprised of two HIV-specific fusion polypeptides capable of binding an HIV viral particle. The capability of the HIV-specific fusion proteins of the invention to bind an HIV viral particle and to block infectivity are measured by methods known in the art, e.g., for example, by a viral infectivity assay, using viruses that express luciferase or another reporter gene to provide an IC50 estimate, as described in Brandt et al. (2002) J. Biol. Chem. 277(19):17291-17299, herein specifically incorporated by reference in its entirety.

In a seventh aspect, the invention features a HIV-specific fusion polypeptide of the invention wherein either or both of the (i) one or more domains of CCR or (ii) CR is (are) replaced with one or more domains which comprise a variable region of an immunoglobulin heavy chain (V_{H}), or a fragment or derivative thereof, and a variable region of an immunoglobulin light chain (V_{L}), or a fragment or derivative thereof. In this aspect of the invention, the one or more variable region component(s) (V_{H}-V_{L}) is (are) immunospecific for a viral protein which interacts with the replaced cellular receptor or co-receptor component. For example, in one embodiment, a CR or CRR domain is replaced with an V_{H}-V_{L} domain immunospecific for gp120. An immunoglobulin fragment specific for gp120 capable of replacing a CCR or CR component is an example of a domain functionally equivalent to the replaced component.

In an eighth aspect, the invention features a nucleic acid sequence encoding a HIV-specific fusion polypeptide, encoding (i) one or more domains which comprise a cellular co-receptor protein, or a fragment or derivative thereof, or one or more V_{H}-V_{L} domains; (ii) one or more domains which comprise a cellular receptor protein, or a fragment or derivative thereof, or one or more V_{H}-V_{L} domains; and optionally (iii) a fusion component, and (iv) one or more domains of a viral protein, or a fragment or derivative thereof.

In a ninth aspect, the invention features therapeutic methods for the treatment of HIV infection comprising administering a therapeutically effective amount of an HIV-specific fusion protein of the invention to a subject in need thereof. The therapeutic methods of the invention may be used to prevent HIV infection in a person at risk for or believed to be at risk for HIV infection. The invention further encompasses therapeutic methods for inhibiting the progression of HIV infection to AIDS.

Accordingly, in a tenth aspect, the invention features pharmaceutical compositions comprising an HIV-specific fusion protein of the invention with a pharmaceutically acceptable carrier. Such pharmaceutical compositions may comprise HIV-specific fusion proteins or nucleic acids encoding HIV-specific fusion proteins.

In an eleventh aspect, the invention features diagnostic and prognostic methods, as well as kits for detecting, quantitating, and/or monitoring HIV with the use of the HIV-specific fusion protein of the invention.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

Definitions

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “multimerizing component” is meant a component which allows a single polypeptide to form a multimer with one or more other polypeptides. Preferably, the multimeric protein is a dimer, but the term “HIV-specific fusion protein” encompasses olimers such as dimers, trimers, tetramers, etc. In a specific embodiment, the multimerizing component comprises a human immunoglobulin derived domain. In more specific embodiments, the immunoglobulin derived domain may be selected from the group consisting of the Fc domain of IgG, the heavy chain of IgG, and the light chain of IgG. The Fc domain of IgG may be selected from IgG1, IgG2, IgG3, and IgG4, and any allotype within each isotype group. In one embodiment, the multimerizing component may be an Fc domain from IgG1 from which the first three to five amino acids are removed and or replaced, for example, the first six amino acids of the Fc region of IgG1 (EPKSCD) (SEQ ID NO:10) are altered to SGD ((“Fc(ΔC1)”). Further embodiments encompass an Fc region from IgG4 in containing a serine to proline change, for example, S10P, and/or other alterations, mutations, deletions, or additions which improve stability or confer a desired characteristic.

The term “spacer” or “linker” means one or more molecules, e.g., nucleic acids or amino acids, which may be inserted between one or more component domains. For example, spacer sequences may be used to provide a restriction site between components for ease of manipulation. A spacer may also be provided to enhance expression of the fusion protein from a host cell, to decrease steric hindrance such that the component may assume its optimal tertiary structure and/or interact appropriately with its target molecule. For spacers and methods of identifying desirable spacers, see, for example, George et al. (2003) Protein Engineering 15:871-879, herein specifically incorporated by reference.

An “HIV-specific” fusion protein of the invention consists of two or more fusion polypeptides of the invention, and is capable of trapping an HIV particle such that the ability of the HIV viral particle to infect a cell is blocked. By “HIV-specific” is meant that the fusion protein of the invention has an affinity for HIV that is ten-fold higher than for another virus, such as for example, MLV, and is exhibits an ability to block HIV infectivity, as measured, for example, by the method of Brandt et al. (2002) supra.

As used herein, the term “HIV infection” generally encompasses infection of a host, particularly a human host, by the human immunodeficiency virus (HIV) family of retroviruses including, but not limited to, HIV I, HIV II, HIV III (also known as HTLV-III, LAV-1, LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as an HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., contact with HIV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HIV infection” may also encompass treating a person who has not been diagnosed as having HIV infection but is believed to be at risk of infection by HIV.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

By the term “functionally equivalent” is meant a component capable of functioning similarly to the reference component. For example, a component that is functionally equivalent to a CCR component may be an immunoglobulin fragment that is capable of binding gp120 with the same functionality as a CCR, such as CCR5, to achieve the same purpose as CCR.

Generation of Antibodies to HIV Proteins

In one embodiment, a fusion polypeptide of the invention comprises one or more immunoglobulin variable regions isolated from antibodies generated against a selected target viral protein. The term “antibody” as used herein refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, etc.). Typically, the antigen-binding region of an antibody will be the most critical in determining specificity and affinity of binding.

An exemplary immunoglobulin (antibody) structural unit comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies (e.g., single chain Fv)(scFv) or those identified using phase display libraries (see, for example, McCafferty et al. (1990) Nature 348:552-554).

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion proteins and methods of the instant invention. Also, transgenic mice, or other organisms such as other mammals, may be used to express human or humanized antibodies. Alternatively, phage display technology can be used to identify antibodies and heteromeric Fab fragments that specifically bind to selected antigens.

Antibody Screening and Selection

Screening and selection of preferred antibodies can be conducted by a variety of methods know n to the art. Initial screening for the presence of monoclonal antibodies specific to a target antigen may be conducted through the use of ELISA-based methods, for example. A secondary screen is preferably conducted to identify and select a desired monoclonal antibody for use in construction of the HIV-specific fusion polypeptides of the invention. Secondary screening may be conducted with any suitable method known to the art. One preferred method, termed “Biosensor Modification-Assisted Profiling” (“BioMAP”) is described in co-pending U.S. Ser. No. 60/423,017 filed 1 Nov. 2002, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody:antigen interactions.

Nucleic Acid Constructs

Individual components of the HIV-specific fusion polypeptides of the invention may be constructed by molecular biological methods known to the art with the instructions provided by the instant specification. These components are selected from a cellular co-receptor protein, such as, for example, CCR5 or CXCR4; a cellular receptor protein, such as, for example, CD4, one or both of which components may be substituted with a lectin-binding receptor such as DC-SIGN; a multimerizing component; a viral protein or fragment thereof; and a variable region of an immunoglobulin heavy chain (V_{H}) or a fragment or derivative thereof, and a variable region of an immunoglobulin light chain (V_{L}), or a fragment or derivative thereof. Encompassed by the invention are components functionally equivalent to CCR5, CXCR4, CD4, etc. Amino acid sequence derivatives of CCR5, CXCR4, CD4, etc., may also be prepared by creating mutations in the encoding nucleic acid molecules. Such variants include, for example, deletions from, or insertions or substitutions of, amino acid residues within the naturally occurring amino acid sequence. Any combination of deletion, insertion, and substitution may be made to arrive at a final construct, provided that the final construct possesses the functionality of the native component in binding an HIV viral particle.

V_{L} and V_{H} domains. After identification and selection of antibodies exhibiting desired binding characteristics, the variable regions of the heavy chain and light chains of each antibody is isolated, amplified, cloned and sequenced. Modifications may be made to the V_{H} and V_{L} nucleotide sequences, including additions of nucleotide sequences encoding amino acids and/or carrying restriction sites, deletions of nucleotide sequences encoding amino acids, or substitutions of nucleotides sequences encoding amino acids.

Specific embodiments of the HIV-specific fusion polypeptides of the invention comprise a multimerizing component which allows the fusion polypeptides of the invention to associate, e.g., as multimers, preferably dimers. Preferably, the multimerizing component comprises an immunoglobulin derived domain. Suitable multimerizing components are sequences encoding an immunoglobulin heavy chain hinge region (Takahashi et al. (1982) Cell 29:671-679); immunoglobulin gene sequences, and portions thereof.

The nucleic acid constructs of the invention are inserted into an expression vector by methods known to the art, wherein the nucleic acid molecule is operatively linked to an expression control sequence. Also provided is a host-vector system for the production of a fusion protein of the invention, which comprises the expression vector of the invention which has been introduced into a host cell suitable for expression of the fusion polypeptide. The suitable host cell may be a bacterial cell such as E. coli, a yeast cell, such as Pichia pastoris, an insect cell, such as Spodoptera frugiperda, or a mammalian cell, such as a COS, CHO, 293, BHK or NS0cell.

The invention further encompasses methods for producing the HIV-specific fusion proteins of the invention by growing cells transformed with an expression vector under conditions permitting production of the HIV-specific fusion proteins and recovery of the fusion proteins so produced.

The invention further encompasses methods for producing the fusion polypeptides or olimeric proteins of the invention by growing cells transformed with an expression vector under conditions permitting production of the fusion polypeptides and recovery of the olimers formed from the fusion polypeptides. Cells may also be transduced with a recombinant virus comprising the nucleic acid construct of the invention.

The HIV-specific proteins may be purified by any technique, which allows for the subsequent formation of a stable olimeric fusion protein. For example, and not by way of limitation, the fusion protein may be recovered from cells either as soluble polypeptides or as inclusion bodies, from which they may be extracted quantitatively by 8M guanidinium hydrochloride and dialysis. In order to further purify the fusion protein, conventional ion exchange chromatography, hydrophobic interaction chromatography, reverse phase chromatography or gel filtration may be used. The fusion proteins may also be recovered from conditioned media following secretion from eukaryotic or prokaryotic cells.

Cell Selection Methodologies

In one embodiment of the invention, cells expressing a HIV-specific fusion protein of the invention are selected having a desired high production rate. A variety of selection processes known to the art may be used. In one preferred embodiment, the selection process is the “FASTR” methodology described in U.S. Ser No. 20020168702 published 14 Nov. 2002, herein specifically incorporated by reference. The FASTR methodology is a high-throughput screening method for rapid isolation of cells secreting a HIV-specific fusion protein of the invention, by direct screening of the fusion polypeptide or protein.

In one embodiment of the cell selection step of the method of the invention, a cell line expressing a cell surface capture molecule which binds the HIV-specific fusion protein is transfected with a nucleic acid construct encoding a HIV-specific fusion polypeptide, which fusion protein is secreted. A cell expressing the HIV-specific fusion protein on its surface is detected by contacting the cell with a detectable molecule which binds the HIV-specific fusion protein, and the detected cell is isolated. Accordingly, the FASTR methodology is one example of a method for detecting a cell producing a high level of the HIV-specific fusion protein of the invention.

Diagnostic Methods

The compositions of the instant invention may be used diagnostically as well as prognostically. For example, an HIV-specific fusion protein of the invention may be used to detect the presence of HIV in a biological sample to determine if a subject is infected with HIV. Further, An HIV-specific fusion protein of the invention can be used to monitor levels of HIV in a biological sample obtained from a subject, to determine severity of infection, progression of infection, and/or during a clinical study to evaluate treatment efficacy.

Similarly, nucleic acids encoding an HIV-specific fusion proteins of the invention may be useful for diagnosis and prognosis of HIV infection and progression to AIDS. Specific nucleic acid constructs may also be useful with oligonucleotide array technology, high density or low density, (e.g., GeneChip™) (see, for example, Gunthand et al. (1998) AIDS Res. Hum. Retroviruses 14:869-876). The HIV-specific fusion proteins of the invention can be used in methods known in the art relating to the localization and activity of HIV, e.g., for imaging HIV, or for delivering a second agent to an HIV viral particle.

Screening and Detection Methods

The HIV-specific fusion proteins of the invention may also be used in in vitro or in vivo screening methods where it is desirable to detect and/or quantify HIV. Screening methods are well known to the art that include cell-free, cell-based, and animal assays. In vitro assays can be either solid state or soluble. Detection of bound or complexed virus may be achieved in a number of ways known to the art, including the use of a label or detectable group capable of identifying a HIV-specific fusion protein which has trapped or otherwise bound an HIV particle. Detectable labels are well-developed in the filed of immunoassays and may generally be used in conjunction with assays using the HIV-specific fusion protein of the invention.

The HIV-specific fusion proteins of the invention may also be directly or indirectly coupled to a label or detectable group when desirable for the purpose it is being used. A wide variety of labels may be used, depending on the sensitivity required, ease of conjugation, stability requirements, available instrumentation, and disposal provisions.

Therapeutic Uses of the HIV Traps of the Invention

The HIV-specific fusion proteins of the invention can be used to inhibit, prevent, and/or reduce HIV infection of cells by, for example, preventing an HIV particle from attaching and entering a cell, and/or promoting the removal an HIV particle from the body of a host subject. The HIV-specific fusion proteins of the invention can be used therapeutically or prophylactically in a subject in need or at risk of HIV infection. For example, they can be used to reduce the viral load from an infected subject. Further, the HIV-specific fusion proteins of the invention can be used to inhibit the progression to AIDS in an HIV infected subject. Still further, the HIV-specific fusion proteins can be used prophylactically, e.g., after exposure or suspected exposure to HIV to prevent infection.

In vitro cell-free or cell-based assays to determine the ability of the HIV trap to bind its target molecule are known to the art. Specifically, for example, HIV entry assays or binding assays have been developed as described in Brandt et al. (2002) supra. Standard methods for measuring in vivo HIV infection and progression to AIDS can be used to determine whether a subject is positively responding to treatment with the HIV-specific fusion protein of the invention. For example, after treatment with an “HIV trap” of the invention, a subject's T cell count can be monitored. A rise in T cells indicates that the subject is benefiting from administration of the HIV trap. Additionally, the “endogenous assay” or “acute infection assay” as described in Levy et al. (1996) Immunology Today 17(5):223 can be used to measure the anti-HIV response of CD8⁺ cells in a subject. For example, in the acute infection assay, CD4⁺ cells from uninfected individuals are acutely infected with HIV and are cultured with CD8⁺ cells from infected individuals at different CD8⁺, CD4⁺ cell ratios. The antiviral effect is determined by the extent of reduction in virus production. These, as well as other methods known to the art, may be used to determine the extent to which the methods of the present invention are effective at inhibiting virus production in a subject.

Methods of Administration

Methods known in the art for the therapeutic delivery of a HIV-specific fusion protein or a nucleic acids encoding a HIV-specific fusion protein of the invention can be used in the methods of the present invention for treating or preventing HIV infection in a subject, e.g., cellular transfection, gene therapy, direct administration with a delivery vehicle or pharmaceutically acceptable carrier, indirect delivery by providing recombinant cells comprising a nucleic acid encoding a HIV-specific fusion protein of the invention, etc.

Various delivery systems are known and can be used to administer the HIV-specific fusion protein of the invention, e.g., encapsulation in liposomes, microparticles, microcapsules, recombinant cells capable of expressing the compound, receptor-mediated endocytosis (see, e.g., Wu et al. (1987) J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105 ). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al. (1991) Proc. Natl. Acad. Sci. USA 88:1864-1868), etc. Alternatively, a nucleic acid can be introduced intracellularly and incorporated within host cell DNA for expression, by homologous recombination.

Cellular Transfection and Gene Therapy

The present invention encompasses the use of nucleic acids encoding the HIV-specific fusion proteins of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.”

In another aspect, the invention provides a method of inhibiting HIV infection in a human comprising transfecting a cell with a nucleic acid encoding a HIV-specific fusion protein of the invention, wherein the nucleic acid comprises an inducible promoter operably linked to the nucleic acid encoding the HIV-specific fusion protein. For gene therapy procedures in the treatment or prevention of human disease, see for example, Van Brunt (1998) Biotechnology 6:1149-1154.

Combination Therapies

In numerous embodiments, the HIV-specific fusion proteins of the present invention may be administered in combination with one or more additional compounds or therapies. For example, multiple fusion proteins can be co-administered, or one or more fusion proteins can be administered in conjunction with one or more therapeutic compounds. For example, the other therapeutic agent is one used to prevent or treat HIV infection, or an agent used to treat an opportunistic infection associated with HIV infection. For example, a suitable therapeutic agent for use in combination with the HIV-specific fusion protein of the invention may include protease inhibitors, antiretroviral nucleosides, fusion inhibitors, entry inhibitors, as well as other anti-viral agents effective to treat or inhibit HIV infection, e.g., zidovudine, interferon, AZT, as well as antibiotics such as acyclovir.

Pharmaceutical Compositions

The present invention also provides pharmaceutical compositions comprising a HIV-specific fusion protein of the invention and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the HIV-specific fusion proteins of the invention which will be effective in the treatment of an HIV-related condition or disease can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 1-20 mg of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Kits

The invention also provides a pharmaceutical pack or kit comprising one or more containers filled with at least one HIV-specific fusion protein the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### HIV-Specific Fusion Polypeptides

DNA sequences encoding the hCD4 Ig domains 1 and 2 and hCD4 Ig domains 3 and 4 were isolated by PCR from a human thymus cDNA library (Clontech cat#7118-1). The hCD4 Ig domains 1 and 2 were amplified using primers with the following sequences: 5′-TTGCGATCGCTAAG AAAGTGGTGCTGGGC-3′(SEQ ID NO:11) and 5′-AATCCGGAAGCTAGCACCACGATGTC-3′ (SEQ ID NO:12). hCD4 Ig domains 3 and 4 were isolated using primers with the following sequences: 5′-TCCGGATTCCAGAAGGCCTCCAGCATAGTC-3′(SEQ ID NO:13) and 5′-TCCGGAGGCGCCGTCACTCAGCAGACACTGCCACATC-3′(SEQ ID NO:14). 5′ and 3′ restriction sites were introduced into each of the primer sequences for use in subcloning the isolated cDNA fragment. The resulting hCD4₃₋₄ fragment was joined with the hCD4₁₋₂ fragment using the introduced restriction site to create hCD4₁₋₄ Human CCR5 N-terminal sequence was obtained by PCR amplification of a human spleen cDNA library (Clontech cat#7125- 1) using primers with the following sequences: 5′-GGCAGATCTGATTATCAAGTGTCAAGTCCA-3′(SEQ ID NO:15) and 5′-CAAACGCGTCAGGAGGCGGGCTGCGATTTG-3′ (SEQ ID NO:16). 5′ and 3′ restriction sites were introduced into each of the primer sequences for use in subcloning the isolated cDNA fragment. The hCD4₁₋₂ d10 deletion clone was created by PCR amplification from CCR5(C→S)-CD4₁₋₂-Fc (SEQ ID NO:1) using overlapping PCR primers (5′-GGGAAGCTGTACAGGTCAGTTCC-ACTGTAGCGATCGCTCCACCACGCGTCAGGAGGCGGGC-3′ (SEQ ID NO:17) and 5′-GCCCGCCTCCTGACGCGTGGTGGAGCGATCGCTACAGTGGAACTGACCTGTACAGCTTCCC-3′ (SEQ ID NO:18) in combination with flanking vector sequence primers with sequences that linked the CCR5 coding sequence with the hCD4d10 sequence. For each of these described PCR fragments, after amplification, the PCR product was gel purified and cloned by topoisomerase mediated TA cloning into the pCR2.1 vector (Invitrogen Topo-TA Cloning Cat# 45-0641) and transfected into E. coli. Alternatively, the PCR product was digested with the relevant restriction enzymes, the fragment was ligated with an appropriate vector and transfected into E. coli. Clones were confirmed by restriction mapping and sequencing.

Traps were constructed by PCR amplifying each of the fragments encoding the various components from the described clones. The primers used for amplification were similar to those described above but contained restriction sites that allowed the ligation of the Trap components to each other and into an expression vector encoding a human Fc gene.

The following amino acid sequences provides examples of fusion polypeptide of the invention: CCR5(C→S)-CD4₁₋₂-Fc (SEQ ID NO:1): Starting at the N-terminus, the construct of SEQ ID NO:1 contains the following components: a single CCR5 N-terminal sequence (1-32) in which the native Cys at position 19 is mutated to an amino acid such as Ser, Ala, or Gly to increase expression (“C→S”); an optional 7 amino acid restriction site linker (bold 33-39); Ig-like domains 1 (40-139) and 2 (140-217) of human CD4 (CD4₁₋₂); an optional 2 amino acid restriction site linker (bold 218-219; followed by human FcΔC1(a) (underlined positions 220-447): DYQVSSPIYDINYYTSEPSQKINVKQIAAR-LLTRGGAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKL TVDKSRWQQGNVFSCSVMHEALHNHYTQKSLS-LSPGK.

Mature CD4₁₋₂/CCR5(C→S)-Fc (SEQ ID NO:2): Starting at the N-terminus of SEQ ID NO:2, the fusion polypeptide contains the following components: an optional 9 amino acid restriction site linker region (bold) is followed by domains 1 (10- 109) and 2 (110-187), an optional 2 amino acid restriction site linker region at 188-189 (bold), a single CCR5 N-terminal sequence (C→S) (190-221); an optional 2 amino acid restriction site linker region at 222-223(bold); and human FcΔC1(a) (underlined positions 224-450):

RSTRGGAIAKKVVLGKKGDTVELTCTASQKKSIQFH WKNSNQ-IKILGN
QGSFLTKGPSKLNDRADSRRSLWDQGNFPLIIKNLKIEDSDTYICEVEDQ
KEEVQLLVFGLTANSDTHLLQGQSLTLTLESPPGSSPSVQCRSPRGKNIQ
GGKTLSVSQLELQDSGTWTCTVLQNQKKVEFKIDIVVLATRDYQVSSPIY
DINYYTSEPSQKINVKQIAARLLSGDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Mature CCR5(C→S)-CD4_{1Δ10-2}-Fc (SEQ ID NO:3): Starting at the N-terminus, the construct of SEQ ID NO:3 contains the following components: a single CCR5 N-terminal sequence (1-32) (C→S); an optional 7 amino acid restriction site linker (bold 33-39); domain 1 with a 10 amino acid deletion at the N-terminus (40-129) and 2 (130-207) of human CD4 (CD4_{1Δ10-2}); an optional 2 amino acid restriction site linker (bold 208-209; followed by human FcΔC1(a) (underlined positions 210-437):

DYQVSSPIYDINYYTSEPSQKINVKQIAARLLTRGGAIATVELTCTASQK
KSIQFHWKNSN-QIKILGNQGSFLTKGPSKLNDRADSRRSLWDQGNFPLI
IKNLKIEDSDTYICEVEDQKEEVQLLVFGLTANSDTHLLQGQSLTLTLES
PPGSSPSVQCRSPRGKNIQGGKTLSVSQLELQDSGTWTCTVLQNQKKVEF
KIDIVVLASGDKTHTCPPCPAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKL
TVDKSRWQQGNVFSCSVMHEALHNHYTQKSLSLSPGK,

Mature CCR5(C→S)-CD4₁₋₄-Fc (SEQ ID NO:4): Starting at the N-terminus, the construct of SEQ ID NO:4 contains the following components: a single CCR5 N-terminal sequence (1-32) (C→S); an optional 7 amino acid restriction site linker (bold 33-39); domains 1 and 2 (40-218) of human CD4; an optional 2 amino acid restriction site linker (bold 219-220; domains 3 and 4 (221-391) of human CD4 Ig; an optional 4 amino acid restriction site linker (bold 392-395); followed by human FcΔC1(a) (underlined positions 396-622):

DYQVSSPIYDINYYTSEPSQKINVKQIAARLLTRGGAIAKKVVL-GKKGD
TVELTCTASQKKSIQFHWKNSNQIKILGNQGSFLTKGPSKLNDRADSRRS
LWDQGNFPLIIKNLKIEDSDTYICEVEDQKEEVQLLVFGLTANSDTHLLQ
GQSLTLTLESPPGSSPSVQCRSPRGKNIQGGKTLSVSQLELQDSGTWTCT
VLQNQKKVEFKIDIVVLASGFQKASSIVYKKEGEQVEFSFPLAFTVEKLT
GSGELWWQAERASSSKSWITFDLKNKEVSVKRVTQDPKLQMGKKLPLHLT
LPQALPQYAGSGNLTLALEAKTGKLHQEVNLVVMRATQLQKNLTCEVWGP
TSPKLMLSLKLENKEAKVSKREKAVWVLNPEAGMWQCLLSDGASGDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK.

Mature CCR5(C→S)-CD4_{1Δ10-4}-Fc (SEQ ID NO:5): Starting at the N-terminus, the construct of SEQ ID NO:4 contains the following components: a single CCR5 N-terminal sequence (1-32) (C→S); an optional 7 amino acid restriction site linker (bold 33-39); domain 1 with a 10 amino acid deletion at the N-terminus and 2 (40-208) of human CD4 Ig; an optional 2 amino acid restriction site linker (bold 209-210; domains 3 and 4 (211-381) of human CD4 Ig; an optional 4 amino acid restriction site linker (bold 382-385); followed by human FcΔC1(a) (underlined positions 386-612):

DYQVSSPIYDINYYTSEPSQKINVKQIAARLLTRGGAIATVELTCTASQK
KSIQFHWKNSNQIKILGNQGSFLTKGPSKLNDRADSRRSLWDQGNFPLII
KNLKIEDSDTYICEVEDQKEEVQLLVFGLTANSDTHLLQGQSLTLTLESP
PGSSPSVQCRSPRGKNIQGGKTLSVSQLELQDSGTWTCTVLQNQKKVEFK
IDIVVLASGFQKASSIVYKKEGEQVEFSFPLAFTVEKLTGSGELWWQAER
ASSSKSWITFDLKNKEVSVKRVTQDPKLQMGKKLPLHLTLPQALPQYAGS
GNLTLALEAKTGKLHQEVNLVVMRATQLQKNLTCEVWGPTSPKLMLSLKL
ENKEAKVSKREKAVWVLNPEAGMWQCLLSDGASGDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK.

CCR5(C→S)-CCR5(C→S)-CD4₁₋₂-Fc (SEQ ID NO:6): Starting at the N-terminus, the construct of SEQ ID NO:6 contains the following components: a first CCR5(C→S) peptide (1-32); an optional 2 amino acid restriction site linker (bold 33-34); a second CCR5(C→S) peptide (35-67); an optional 3 amino acid restriction site linker (bold 68-70); domains 1 and 2 (71-249) of human CD4; an optional 2 amino acid restriction site linker (bold 250-251); followed by human FcΔC1(a) (underlined positions 252-479):

DYQVSSPIYDINYYTSEPSQKINVKQIAARLLTRDYQVSSPIYDINYY-T
SEPSQKINVKQIAARLLAIAKKVVLGKKGDTVELTCTASQKKSIQFHWKN
SNQIKILGNQGSFLTKGPSKLNDRADSRRSLWDQGNFPLIIKNLKIEDSD
TYICEVEDQKEEVQLLVFGLTANSDTHLLQGQSLTLTLESPPGSSPSVQC
RSPRGKNIQGGKTLSVSQLELQDSGTWTCTVLQNQKKVEFKIDIVVLASG
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

CCR5(C→S)-CD4₁₋₂-Fc-CCR5(C→S) (SEQ ID NO:7): Starting at the N-terminus, the construct of SEQ ID NO:7 contains the following components: a first CCR5(C→S) peptide (1-32); an optional 7 amino acid restriction site linker (bold 33-39); domains 1 and 2 (40-218) of human CD4; an optional 2 amino acid restriction site linker (bold 219-220); human FcΔC1(a) (underlined positions 221-448); an optional 2 amino acid restriction site linker (bold 449-450); a second CCR5(C→S) peptide at 451-482; and an optional 2 amino acid restriction site linker (bold 483-484):

DYQVSSPIYDINYYTSEPSQKINVKQIAARLLTRGGAIAKKVVLGKKGDT
VELTCTASQKKSIQFHWKNSNQIKILGNQGSFLTKGPSKLNDRADSRRSL
WDQGNFPLIIKNLKIEDSDTYICEVEDQKEEVQLLVFGLTANSDTHLLQG
QSLTLTLESPPGSSPSVQCRSPRGKNIQGGKTLSVSQLELQDSGTWTCTV
LQNQKKVEFKIDIVVLASGDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKASAD
YQVSSPIYDINYYTSEPSQKINVKQIAARLLSR.

CD4₁₋₂-Fc-CCR5(C→S) (SEQ ID NO:8): Starting at the N-terminus, the construct of SEQ ID NO:8 contains the following components: an optional 9 amino acid restriction site linker (bold 1-9); domains 1 and 2 (10-188) of human CD4; an optional 2 amino acid restriction site linker (bold 189-190); human FcΔC1(a) (underlined positions 191-418); an optional 2 amino acid restriction site linker (bold 419-420); CCR5(C→S) peptide at 421-452; and an optional 2 amino acid restriction site linker (bold 453-454):

RSTRGGAIAKKVVLGKKGDTVELTCTASQKKSIQFHWKNSNQIKILGNQG
SFLTK-GPSKLNDRADSRRSLWDQGNFPLIIKNLKIEDSDTYICEVEDQK
EEVQLLVFGLTANSDTHLLQGQSLTLTLESPPGSSPSVQCRSPRGKNIQG
GKTLSVSQLELQDSGTWTCTVLQNQKKVEFKIDIVVLASGDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGKASADYQVSSPIYDINYYTSEPSQKINVKQIAAR
LLSR.

### Example 2

### Expression of HIV-Specific Dimeric Fusion Polypeptides

HIV-specific fusion polypeptides were secreted as dimers (“HIV traps”) through association of the Fc components when transiently expressed in CHO-K1 cells. Two ug of an expression vector encoding the indicated HIV Trap was transfected into one well of a 6-well plate using Lipofectamine (Invitrogen cat# 18324-020) in 1 ml Optimem-1 media (Gibco cat# 31985-070) following the manufacturer's protocol. Five hours post transfection an additional 1 ml of optimem-1+10% fetal calf serum was added per well. At 24 hours post transfection cell media was changed to CHO-SFM-II (Gibco cat# 31033-020) with 10 nM sodium butyrate. Supernatents were collected 72 hours post media change and trap expression levels were analyzed by non-reducing SDS-PAGE. Twenty ul of cell supernatent were loaded on a 4-12% gradient tris-glycine gel (Invitrogen cat# EC6038BOX).

Following electrophoresis proteins were electro-transferred to an Immobilon-P membrane and detected using an anti-human Fc HRP conjugated antibody (Promega anti-human H+L, cat# W403B, 1:20,000 dilution) using standard Western blot methods.

Results: Mutation of the Cys residue at CCR5 position 19 to Ser [CCR5(C→S)-CD4₁₋₂-Fc] (SEQ ID NO:1)was shown to increase expression of the fusion polypeptide relative to CCR5-CD4₁₋₂-Fc (SEQ ID NO:9) from an initial level of 0.1-0.3 ug/ml to 5-7.5 ug/ml. The CD4₁₋₂-CCR5(C→S)-Fc fusion polypeptide was shown to increase expression from an approximate level of 0.8-1 ug/ml to 5 ug/ml with the addition of the C→S mutation. CD4₁₋₂-Fc-CCR5(C→S) (SEQ ID NO:8) and CCR5(C→S)-CD4₁₋₂-Fc-CCR5(C→S) (SEQ ID NO:7) format traps were shown to express at approximate levels of 5 to 6 ug/ml. Deletion of 10 amino acids of the amino terminus of Ig-like domain 1 of CD4, as well as an HIV trap containing the full length extracellular region of hCD4 [CCR5(C→S)-4₁₋₄-Fc (SEQ ID NO:4)], had expression levels of less than 0.1 ug/ml.

What is claimed is:

1. An isolated nucleic acid encoding a fusion polypeptide, wherein the fusion polypeptide comprises:
(a) one or more domains which comprise a cellular co-receptor protein, or a fragment, derivative or functional equivalent thereof (CCR);
(b) one or more domains which comprise a cellular receptor protein, or a fragment, derivative, or functional equivalent thereof (CR); and optionally
(c) a fusion component (FC), and
(d) one or more domains of a viral protein, or a fragment or derivative thereof (VP).

2. The isolated nucleic acid of claim 1, wherein CCR is one or more protein(s) selected from the group consisting of (i) human CCR5, or a fragment, derivative or functional equivalent thereof, (ii) human CXCR4, or a fragment, derivative or functional equivalent thereof, and (iii) a lectin-binding receptor.

3. The isolated nucleic acid of claim 2, wherein a functional equivalent of (i) or (ii) is an immunoglobulin variable region or fragment thereof immunospecific for a viral protein which interacts with (i) or (ii).

4. The isolated nucleic acid of claim 2, wherein the fusion polypeptide comprises more than on CCR domain which are the same or different proteins.

5. The isolated nucleic acid of claim 1, wherein CR is one or more protein(s) selected from the group consisting of (i) human CD4, or a fragment, derivative or functional equivalent thereof, and (ii) a lectin-binding receptor.

6. The isolated nucleic acid of claim 5, wherein a functional equivalent is an immunoglobulin variable region or fragment thereof immunospecific for a viral protein which interacts with CD4.

7. The isolated nucleic acid of claim 5, wherein the human CD4 fragment comprises Ig-like domain 1, or a fragment or derivative thereof capable of binding gp120.

8. The isolated nucleic acid of claim 5, wherein the fusion polypeptide comprises more than on CR domain which are the same or different proteins.

9. The isolated nucleic acid of claim 1, wherein FC is selected from the group consisting of a multimerizing component, fusion partner, a targeting protein, a serum protein, or a molecule capable of binding a serum protein.

10. The isolated nucleic acid of claim 9, wherein the multimerizing component is selected from the group consisting of (i) an immunoglobulin-derived domain, (ii) a cleavable region (C-region), (ii) an amino acid sequence between 1 to about 500 amino acids in length, optionally comprising at least one cysteine residue, (iii) a leucine zipper, (iv) a helix loop motif, and (v) a coil-coil motif.

11. The isolated nucleic acid of claim 10, wherein the immunoglobulin-derived domain is selected from the group consisting of the Fc domain of IgG, the heavy chain of IgG, and the light chain of IgG.

12. The isolated nucleic acid of claim 11, wherein the Fc domain of IgG is human FcΔ1(a).

13. The isolated nucleic acid of claim 1, wherein VP is a viral receptor.

14. The isolated nucleic acid of claim 13, wherein the viral receptor protein is gp41 or a fragment or derivative thereof.

15. A fusion polypeptide encoded by the isolated nucleic acid of claim 1.

16. The fusion polypeptide of claim 15, selected from the group consisting of SEQ ID NO:1-9.

17. A method of producing a fusion protein, comprising culturing a host cell transfected with a vector comprising the nucleic acid of claim 1, under conditions suitable for expression of the protein from the host cell, and recovering the fusion protein so produced.

18. The fusion polypeptide of claim 15 which is a dimer.

19. A fusion polypeptide, comprising:
(a) one or more domains which comprise a cellular co-receptor protein, or a fragment, derivative or functional equivalent thereof (CCR);
(b) one or more domains which comprise a cellular receptor protein, or a fragment, derivative, or functional equivalent thereof (CR); and optionally
(c) a fusion component (FC), and
(d) one or more domains of a viral protein, or a fragment or derivative thereof (VP).

20. The fusion polypeptide of claim 19, wherein CCR is one or more protein(s) selected from the group consisting of (i) human CCR5, or a fragment, derivative or functional equivalent thereof, (ii) human CXCR4, or a fragment, derivative or functional equivalent thereof, and (iii) a lectin-binding receptor.

21. The fusion polypeptide of claim 20, wherein a functional equivalent of (i) or (ii) is an immunoglobulin variable region or fragment thereof immunospecific for a viral protein which interacts with (i) or (ii).

22. The fusion polypeptide of claim 20, wherein the fusion polypeptide comprises more than on CCR domain which are the same or different proteins.

23. The fusion polypeptide of claim 19, wherein CR is one or more protein(s) selected from the group consisting of (i) human CD4, or a fragment, derivative or functional equivalent thereof, and (ii) a lectin-binding receptor.

24. The fusion polypeptide of claim 23, wherein a functional equivalent is an immunoglobulin variable region or fragment thereof immunospecific for a viral protein which interacts with CD4.

25. The fusion polypeptide of claim 24, wherein the human CD4 fragment comprises Ig-like domain 1, or a fragment or derivative thereof capable of binding gp120.

26. The fusion polypeptide of claim 23, wherein the fusion polypeptide comprises more than on CR domain which are the same or different proteins.

27. The fusion polypeptide of claim 19, wherein FC is selected from the group consisting of a multimerizing component, fusion partner, a targeting protein, a serum protein, or a molecule capable of binding a serum protein.

28. An HIV-specific protein capable of binding an HIV viral particle and/or blocking the ability of an HIV viral particle to infect a cell comprising two of the fusion proteins of claim 19.

29. A pharmaceutical composition comprising the HIV-specific fusion protein of claim 28 and a pharmaceutically acceptable carrier.

30. A therapeutic method for treating an HIV infection in a subject in need thereof, comprising administering the pharmaceutical composition of claim 29.

31. The therapeutic method of claim 30, wherein the subject is a human suffering from or at risk for infection from HIV.

32. A therapeutic method for reducing a viral load of a patient suffering from HIV infection, comprising administering the pharmaceutical composition of claim 29.


SEQUENCE LISTING (18 sequences)

SEQ ID NO: 1 - protein, 446 aa, Artificial Sequence
  Synthetic
DYQVSSPIYDINYYTSEPSQKINVKQIAARLLTRGGAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 450 aa, Artificial Sequence
  Synthetic
RSTRGGAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLATRDYQVSSPIYDINYYTSEPSQKINVKQIAARLLSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 436 aa, Artificial Sequence
  Synthetic
DYQVSSPIYDINYYTSEPSQKINVKQIAARLLTRGGAIATVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 621 aa, Artificial Sequence [sequence omitted]

SEQ ID NO: 5 - protein, 611 aa, Artificial Sequence
  Synthetic
DYQVSSPIYDINYYTSEPSQKINVKQIAARLLTRGGAIATVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDGASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 476 aa, Artificial Sequence
  Synthetic
DYQVSSPIYDINYYTSEPSQKINVKQIAARLLTRDYQVSSPIYDINYYTSEPSQKINVKQIAARLLAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 483 aa, Artificial Sequence [sequence omitted]

SEQ ID NO: 8 - protein, 453 aa, Artificial Sequence
  Synthetic
RSTRGGAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASADYQVSSPIYDINYYTSEPSQKINVKQIAARLLSR

SEQ ID NO: 9 - protein, 446 aa, Artificial Sequence
  Synthetic
DYQVSSPIYDINYYTSEPCQKINVKQIAARLLTRGGAIAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLASGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 6 aa, Artificial Sequence
  Synthetic
EPKSCD

SEQ ID NO: 11 - DNA, 29 bp, Artificial Sequence
  Primer
ttgcgatcgctaagaaagtggtgctgggc

SEQ ID NO: 12 - DNA, 26 bp, Artificial Sequence
  Primer
aatccggaagctagcaccacgatgtc

SEQ ID NO: 13 - DNA, 30 bp, Artificial Sequence
  Primer
tccggattccagaaggcctccagcatagtc

SEQ ID NO: 14 - DNA, 37 bp, Artificial Sequence
  Primer
tccggaggcgccgtcactcagcagacactgccacatc

SEQ ID NO: 15 - DNA, 30 bp, Artificial Sequence
  Primer
ggcagatctgattatcaagtgtcaagtcca

SEQ ID NO: 16 - DNA, 30 bp, Artificial Sequence
  Primer
caaacgcgtcaggaggcgggctgcgatttg

SEQ ID NO: 17 - DNA, 61 bp, Artificial Sequence
  Primer
gggaagctgtacaggtcagttccactgtagcgatcgctccaccacgcgtcaggaggcgggc

SEQ ID NO: 18 - DNA, 61 bp, Artificial Sequence
  Primer
gcccgcctcctgacgcgtggtggagcgatcgctacagtggaactgacctgtacagcttccc